(19) United States
(12) Patent Application Publication
(10) Pub. No.: US 2026/0000401 A1
(43) Pub. Date: Jan. 1, 2026

(54) ELECTRICAL IMPEDANCE TOMOGRAPHY DEVICE AND SYSTEM

(71) Applicants: Covidien LP, Mansfield, MA (US); TRUSTEE OF DARTMOUTH COLLEGE, Hanover, NH (US)

(72) Inventors: Matthew S. ESCHBACH, North Haven, CT (US); Robert H. KNAPP, North Haven, CT (US); Alexander W. CAULK, North Haven, CT (US); Brian L. HOLDEN, North Haven, CT (US); Ryan J. HALTER, North Haven, CT (US); Harshavardhan DEVARAJ, Lebanon, NH (US); Ethan K. MURPHY, Hanover, NH (US)

(73) Assignees: Covidien LP, Mansfield, MA (US); TRUSTEE OF DARTMOUTH COLLEGE, Hanover, NH (US)

(21) Appl. No.: 18/868,951

(22) PCT Filed: May 23, 2023
(86) PCT No.: PCT/US2023/023166

§ 371 Date: Nov. 25, 2024

Related U.S. Application Data
(60) Provisional application No. 63/347,336, filed on May 31, 2022.

(51) Int. Cl. A61B 17/072; A61B 5/0536

(52) U.S. Cl. CPC A61B 17/07207; A61B 5/0536

(57) ABSTRACT

A surgical system includes a surgical stapler and an electrical impedance tomography (EIT) system. The surgical stapler includes a first jaw having an anvil and a second jaw having a stapler cartridge. The EIT system includes an electrode array including a plurality of electrodes operably coupled to the first jaw and/or the second jaw, and a processor. The processor is configured to control an application of an electrical current across electrodes of the electrode array, measure a voltage difference across electrodes of the electrode array, calculate electrical impedance based on the measured voltage difference, and generate an electrical impedance tomography reconstruction based on the calculated electrical impedance.

### BACKGROUND

### 1. Technical Field

The present disclosure relates to surgical devices. More specifically, the present disclosure relates to surgical devices including an array of electrodes for electrical impedance tomography and a system for reconstructing electrical impedance tomography images.

### 2. Background of Related Art

Surgical staplers are commonly used in numerous open and laparoscopic cancer resection procedures including thoracic, bariatric, and colorectal surgery. For instance, during lung procedures surgical staplers are often used to cut a wedge resection around a lung nodule suspected to be cancerous. However, small lung nodules may be thoracoscopically invisible and impalpable during surgery, requiring different localization methods, such as hook wires, dyes, fiducial markers, contrast media, or radiotracers. These localization methods while often very effective, e.g., success rates of 93.6-97.6% for hook wire via computed tomography (CT) guidance for localization, have certain disadvantages and can fail.

As cancer is being identified earlier, the sizes of the tumors being found are smaller. This can make intra-operatively identifying the tumor difficult. This is especially true in lung cancer. CT images can confirm the presence of cancer but are still not easily translated to the lung geometry as the geometry changes upon deflation during surgery. In addition, cancers might be so small or so deep within the parenchyma that a surgeon cannot identify them using video-assisted thoracic surgery (VATS) or even palpate them during open procedures. Therefore, a method of intraoperatively locating cancerous lung nodules, or malignant tissue generally, is required.

### SUMMARY

The present disclosure provides a surgical system including a surgical stapler and an electrical impedance tomography (EIT) system. The surgical stapler includes an end effector including a first jaw having an anvil and a second jaw having a stapler cartridge. The EIT system includes an electrode array including a plurality of electrodes operably coupled to the first jaw and a processor. The processor is configured to control an application of an electrical current across electrodes of the electrode array, measure a voltage difference across electrodes of the electrode array, calculate electrical impedance based on the measured voltage difference, and generate an electrical impedance tomography reconstruction based on the calculated electrical impedance. In an aspect, the electrode array may include sixty electrodes.

In an aspect, each electrode of the electrode array may be about 3 mm long and about 1 mm wide. As used herein, the term “about” denotes+5% of the numerical value.

In an aspect, the electrodes of the electrode array may be arranged in four columns and fifteen rows.

In an aspect, the electrodes of the electrode array may be grouped into four distinct sets of ten electrodes.

In an aspect, the processor may be configured to filter the calculated electrical impedance by removing noisy patterns prior to generating the electrical impedance tomography reconstruction.

In an aspect, the generated electrical impedance tomography reconstruction may include conductive inclusions and resistive inclusions, wherein the conductive inclusions are visually distinguishable from the resistive inclusions.

In an aspect, the processor may be configured to detect tumor margins based on the electrical impedance tomography reconstruction.

In an aspect, the processor may be configured to control an application of the electrical current at a frequency from about 10 kHz to about 80 KHz.

In another aspect of the disclosure a powered surgical instrument is provided. The powered surgical instrument includes an end effector including a first jaw and a second jaw and an electrical impedance tomography system. The electrical impedance tomography system includes a first electrode array including a plurality of electrodes operably coupled to the first jaw, a second electrode array including a plurality of electrodes operably coupled to the second jaw, and a processor. The processor is configured to control an application of an electrical current across any two electrodes of the first electrode array and the second electrode array, measure a voltage difference across the first electrode array and the second electrode array (e.g., any other two electrodes of the first electrode array and the second electrode array) or between any two electrodes of the first electrode array or the second electrode array, calculate electrical impedance based on the measured voltage difference, and generate an electrical impedance tomography reconstruction based on the calculated electrical impedance.

In an aspect, the first electrode array may include sixty electrodes and the second electrode array includes eight electrodes.

In an aspect, each electrode of the first electrode array may be about 3 mm long and about 1 mm wide and each electrode of the second electrode array may be about 3 mm long and about 3 mm wide.

In an aspect, the electrodes of the first electrode array may be arranged in four columns and fifteen rows and the electrodes of the second electrode array may be arranged in two columns and four rows.

In an aspect, at least a portion of the plurality of electrodes of the first electrode array may be grouped into four distinct sets of ten electrodes, each set of ten electrodes being paired with eight electrodes of the second electrode array.

In an aspect, the processor may be configured to filter the calculated electrical impedance by removing noisy patterns prior to generating the electrical impedance tomography reconstruction.

In an aspect, the generated electrical impedance tomography reconstruction may include conductive inclusions and resistive inclusions, wherein the conductive inclusions are optically distinguishable from the resistive inclusions.

In an aspect, the processor may be configured to detect tumor margins based on the electrical impedance tomography reconstruction.

In an aspect, the processor may be configured to control an application of the electrical current across the first electrode array and the second electrode array at a frequency from about 10 kHz to about 80 kHz.

In another aspect of the present disclosure a surgical system includes a powered surgical instrument, an electrical impedance tomography system, and a display. The powered surgical instrument includes a first jaw and a second jaw. The electrical impedance tomography system includes a first electrode array including a plurality of electrodes operably coupled to the first jaw, a second electrode array including a plurality of electrodes operably coupled to the second jaw, and a processor. The processor is configured to control an application of an electrical current across the first electrode array and the second electrode array, measure a voltage difference across the first electrode array and the second electrode array, calculate electrical impedance based on the measured voltage difference, and generate an electrical impedance tomography reconstruction based on the calculated electrical impedance. The display is operably couped to the electrical impedance tomography system and configured to display the electrical impedance tomography reconstruction.

In an aspect, the processor may be configured to control an application of the electrical current across the first electrode array and the second electrode array at a frequency from about 10 kHz to about 80 kHz.

### BRIEF DESCRIPTION OF THE DRAWINGS

Embodiments of the present disclosure are described herein with reference to the accompanying drawings, wherein:

FIG. 1 is a surgical system including an electrical impedance tomography system and a surgical instrument according to an embodiment of the present disclosure;

FIG. 2 is a perspective view of an exemplary surgical instrument of FIG. 1 in the form of a powered surgical stapler including a handle assembly, an adapter assembly, and an end effector, according to an embodiment of the present disclosure;

FIG. 3 is a schematic diagram of the handle assembly, the adapter assembly, and the end effector of the surgical instrument of FIG. 2;

FIG. 4 is a top view of a bottom electrode array of the surgical instrument of FIG. 2;

FIG. 5 is a top view of a top electrode array of the surgical instrument of FIG. 2;

FIG. 6 is an electrical impedance tomography reconstruction displayable on a graphical user interface (GUI) of the surgical system of FIG. 1;

FIG. 7 is a diagram of the bottom electrode array of the surgical instrument of FIG. 2 relative to simulated grid marks for segmenting, in accordance with an aspect of the present disclosure;

FIG. 8A is a graph illustrating area-under-the-curve (AUC) calculations across all difference-EIT reconstructions from simulated tests using 2% simulated noise and varying the sensitivity threshold from 1/50 to 1/1000 on top and bottom electrodes;

FIG. 8B is a graph illustrating AUC calculations across all difference-EIT reconstructions from simulated tests using 2% simulated noise and varying the sensitivity threshold from 1/50 to 1/1000 on bottom-only electrodes;

FIG. 9A is a graph illustrating AUC calculations across all difference-EIT reconstructions from simulated tests varying the voltage noise while using 2% simulated noise and a sensitivity threshold of 1/50;

FIG. 9B is a graph illustrating AUC calculations across all difference-EIT reconstructions from simulated tests varying the reference z-height while using 2% simulated noise and a sensitivity threshold of 1/50;

FIG. 10A is a graph illustrating AUC calculations across all available difference-EIT reconstructions from simulated tests analyzed across contrasts using 2% voltage noise and a sensitivity threshold of 1/50;

FIG. 10B is a graph illustrating AUC calculations across all available difference-EIT reconstructions from simulated tests analyzed across size using 2% voltage noise and a sensitivity threshold of 1/50; and

FIG. 10C is a graph illustrating AUC calculations across all available difference-EIT reconstructions from simulated tests analyzed across x/y-distance from electrodes to the inclusion center using 2% voltage noise and a sensitivity threshold of 1/50.

### DETAILED DESCRIPTION OF EMBODIMENTS

Embodiments of the presently disclosed surgical devices, and adapter assemblies for surgical devices and/or handle assemblies are described in detail with reference to the drawings, in which like reference numerals designate identical or corresponding elements in each of the several views. As used herein the term “distal” refers to that portion of the surgical instrument, or component thereof, farther from the user, while the term “proximal” refers to that portion of the surgical instrument, or component thereof, closer to the user.

This disclosure provides for the incorporation of electrical impedance tomography (EIT) with a surgical device, such as a laparoscopic surgical stapler, surgical probe, surgical grasper, or any other surgical device, for generating electrical impedance tomography reconstructions or other electrical impedance data sets (e.g., EIT mapping) and enabling intraoperative identification of nodule (e.g., tumor) margins. Although the detection of tumor margins is described, it is contemplated that the embodiments may be utilized to detect disease states and/or critical structures as well. In embodiments, other surgical instruments may incorporate the EIT system according to the present disclosure including, but not limited to, graspers, vessel sealers (bipolar or ultrasonic), wands, probes, and the like.

EIT estimates electrical properties from many combinations of impedance measurements recorded from electrodes placed on the tissue. Electrical impedance (Z) may be calculated using a 4-point measurement, i.e., one can apply electrical current (I) across two electrodes and measure the voltage difference (V) from two other electrodes (Z=V/I). The significant cellular and extracellular differences between benign and malignant tissues or other tissues and nodules result in significant electrical impedance differences.

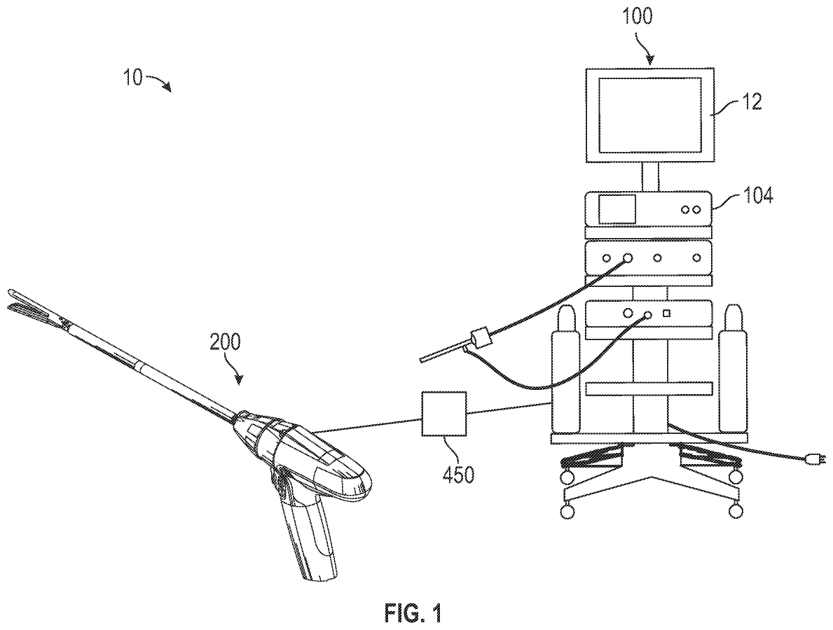

FIG. 1 shows a surgical system 10 including a computing device 100 configured to communicate with one or more surgical devices (e.g., surgical instrument 200). The computing device 100 includes one or more displays 12 configured to output information pertaining to the devices and the surgical setting as graphical user interfaces on the one or more displays 12. The displays 12 may be any suitable monitor, an augmented or virtual reality headset, a heads-up display, a projector, etc. In embodiments, the displays 12 may also be touchscreens. The system 10 also includes an interface device 104, which is configured to communicate with a surgical instrument 200, which may include modular surgical staplers (e.g., a linear stapler, circular stapler, etc.) which may be powered or non-powered. The interface device 104 is further configured to receive device information from the surgical instrument 200 and to process device information for display on one or more displays 12.

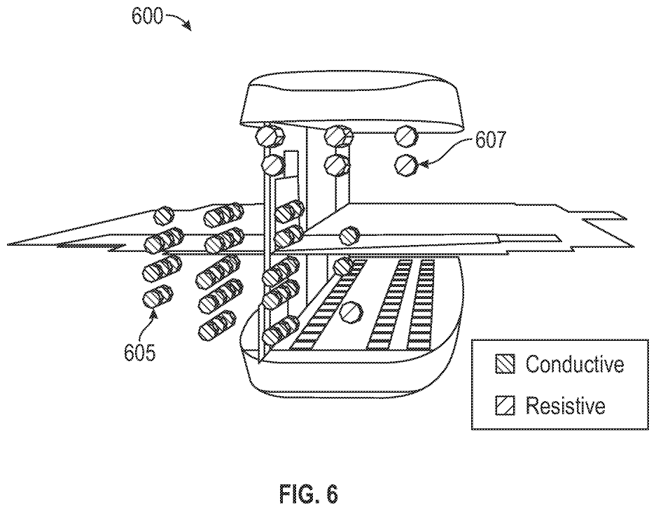

The surgical system 10 further includes an electrical impedance tomography (EIT) system 450 that is configured to generate electrical impedance tomography reconstructions 600 (FIG. 6) of portions of a surgical site for display on the display 12. The EIT system 450 may be entirely incorporated into the surgical instrument 200 and configured to output data to the computing device 100 for additional processing and display on display 12. Alternatively, the EIT system 450 may include one or more components incorporated into the surgical instrument 200 and one or more components separate from the surgical instrument 200.

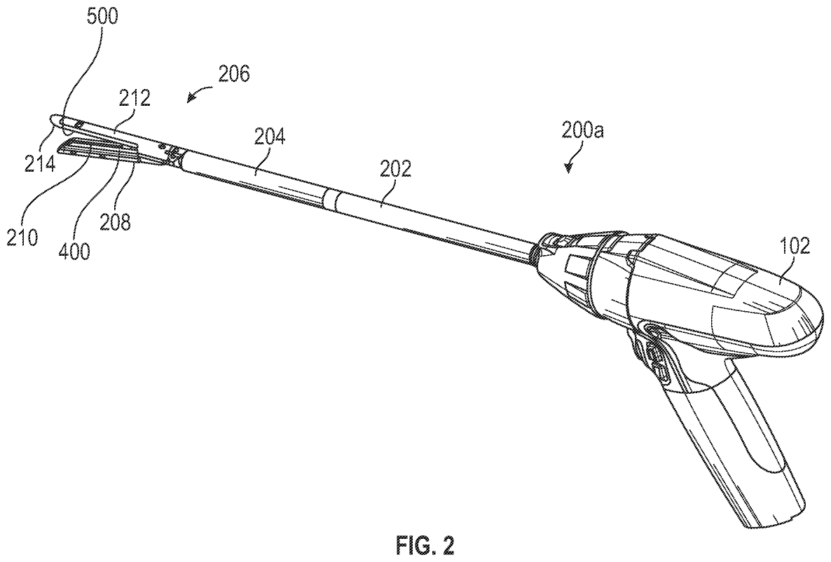

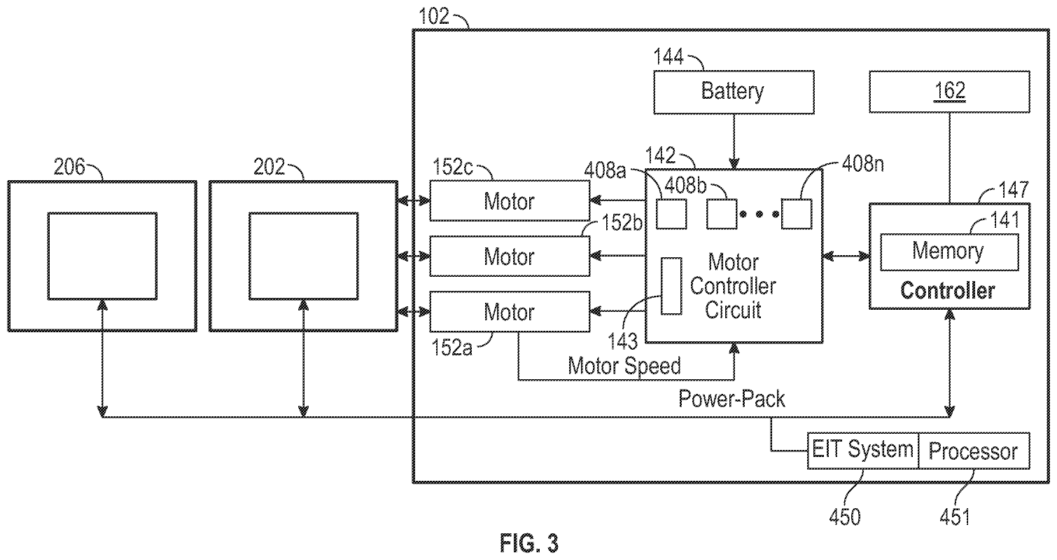

In aspects, the surgical instrument 200 may be a powered surgical instrument such as powered surgical stapler 200a (FIGS. 2 and 3). FIG. 2 illustrates the components of an example powered surgical stapler 200a which includes a power platform, i.e., a handle assembly 102 including one or more motors, a power source, a main controller, storage device, transmitter/receiver, etc. The powered surgical stapler 200a also includes a linear adapter 202 configured to connect the handle assembly 102 to a loading unit 204 including an end effector 206 having a first jaw 208 having a stapler cartridge 210 and a second jaw 212 having an anvil 214. The linear adapter 202 includes various mechanical linkages coupling the end effector 206 with the handle assembly 102 enabling actuation of the end effector 206 to perform various functions, e.g., clamp, staple, cut. For further details regarding the construction and operation of the linear stapler components, reference may be made to U.S. Pat. No. 9,839,425, filed on Mar. 30, 2015, the entire contents of which being incorporated by reference herein.

With reference to FIG. 3, the handle assembly 102 includes a main controller circuit board 142, a rechargeable battery 144 configured to supply power to any of the electrical components of handle assembly 102, and a plurality of motors, e.g., a first motor 152a, a second motor 152b, and a third motor 152c coupled to the battery 144. The handle assembly 102 also includes a display 146. In embodiments, the motors 152a, 152b, 152c may be coupled to any suitable power source configured to provide electrical energy to the motors 152a, 152b, 152c, such as an AC/DC transformer. Each of the motors 152a, 152b, 152c is coupled to a motor controller 143 which controls the operation of the corresponding motors 152a, 152b, 152c including the flow of electrical energy from the battery 144 to the motors 152a, 152b, 152c. A main controller 147 is provided that controls the handle assembly 102. The main controller 147 is configured to execute software instructions embodying algorithms, such as clamping, stapling, and cutting algorithms which control operation of the handle assembly 102.

The motor controller 143 includes a plurality of sensors (not shown) configured to measure operational states of the motors 152a, 152b, 152c and the battery 144. The sensors may include a strain gauge and may also include voltage sensors, current sensors, temperature sensors, telemetry sensors, optical sensors, and combinations thereof. The strain gauge may be disposed within the linear adapter 202. The sensors may measure voltage, current, and other electrical properties of the electrical energy supplied by the battery 144. The sensors may also measure angular velocity (e.g., rotational speed) as revolutions per minute (RPM), torque, temperature, current draw, and other operational properties of the motors 152a, 152b, 152c. The sensors may also include an encoder configured to count revolutions or other indicators of the motors 152a, 152b, 152c, which is then used by the main controller 147 to calculate linear movement of components movable by the motors 152a, 152b, 152c. Angular velocity may be determined by measuring the rotation of the motors 152a, 152b, 152c or a drive shaft (not shown) coupled thereto and rotatable by the motors 152a, 152b, 152c. The position of various axially movable drive shafts may also be determined by using various linear sensors disposed in or in proximity to the shafts or extrapolated from the RPM measurements. In embodiments, torque may be calculated based on the regulated current draw of the motors 152a, 152b, 152c at a constant RPM. In further embodiments, the motor controller 143 and/or the main controller 147 may measure time and process the above-described values as a function of time, including integration and/or differentiation, e.g., to determine the rate of change in the measured values. The main controller 147 is also configured to determine distance traveled of various components of the linear adapter 202 and/or the end effector 206 by counting revolutions of the motors 152a, 152b, 152c.

The motor controller 143 is coupled to the main controller 147, which includes a plurality of inputs and outputs for interfacing with the motor controller 143. In particular, the main controller 147 receives measured sensor signals from the motor controller 143 regarding operational status of the motors 152a, 152b, 152c and the battery 144 and, in turn, outputs control signals to the motor controller 143 to control the operation of the motors 152a, 152b, 152c based on the sensor readings and specific algorithm instructions. The main controller 147 is also configured to accept a plurality of user inputs from a user interface (e.g., switches, buttons, touch screen, etc.) coupled to the main controller 147.

The main controller 147 is also coupled to a memory 141. The memory 141 may include volatile (e.g., RAM) and non-volatile storage configured to store data, including software instructions for operating the handle assembly 102. The main controller 147 is also coupled to a strain gauge (not shown) using a wired or a wireless connection and is configured to receive strain measurements from the strain gauge, and also impedance information, which are used during operation of the handle assembly 102.

The handle assembly 102 includes a plurality of motors 152a, 152b, 152c each including a respective motor shaft (not explicitly shown) extending therefrom and configured to drive a respective transmission assembly. Rotation of the motor shafts by the respective motors functions to drive shafts and/or gear components of linear adapter 202 in order to perform the various operations of handle assembly 102. In particular, motors 152a, 152b, 152c of handle assembly 102 are configured to drive shafts and/or gear components of linear adapter assembly 202 in order to actuate the end effector 206.

The handle assembly 102 also includes a communication interface 162 configured to connect to the interface device 104 using a wired (e.g., Firewire®, USB®, Serial RS232®, Serial RS485®, USART®, Ethernet®, etc.) or wireless (e.g., Bluetooth®, ANT3®, KNX®, ZWave®, X10® Wireless USB®, IrDAR, Nanonet®, Tiny OS®, ZigBee®, 802.11 IEEE, and other radio, infrared, UHF, VHF communications and the like) connection. The interface device 104 is configured to store the data transmitted thereto by the powered surgical instrument 200 as well as process and analyze the data. The interface device 104 is also connected to other devices, such as the processor 451 of the EIT system 450 (when the processor is external to the surgical instrument 200) and computing device 100.

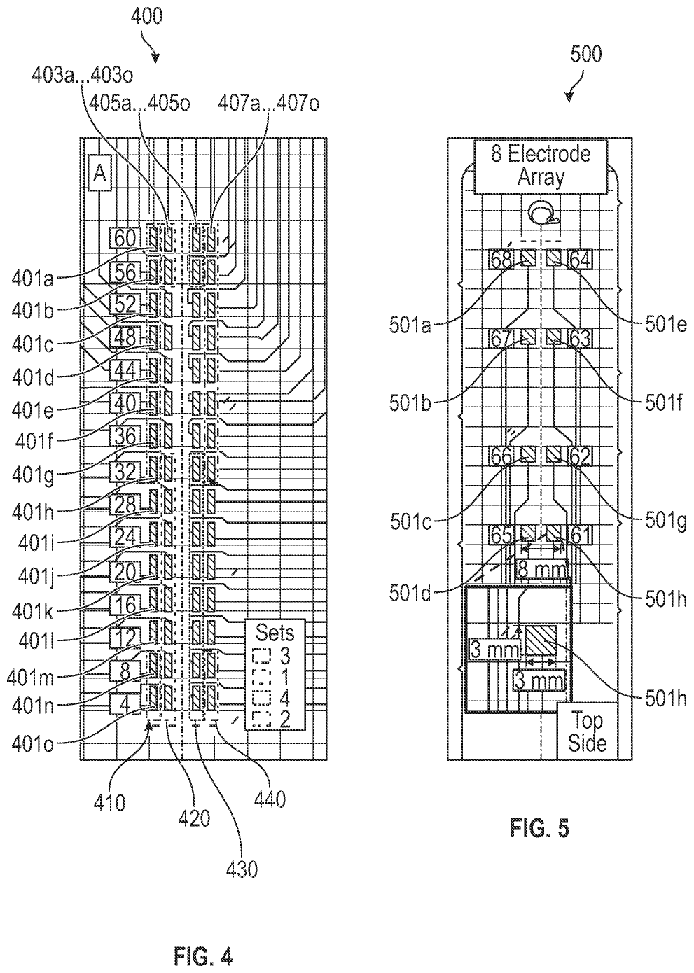

Turning now to FIGS. 4 and 5, the EIT system 450 includes a first electrode array 400, and in aspects, additionally includes a second electrode array 500. The first electrode array 400 is coupled to the first jaw 208 (FIG. 2) of end effector 206, and in aspects that include a second electrode array 500 (FIG. 5), the second electrode array 500 is coupled to the second jaw 212 of the end effector 206. Although described in this particular configuration, the first electrode array 400 and/or the second electrode array 500 may be coupled to a probe (e.g., on a single side of a surgical device). The first electrode array 400 may include any suitable number of electrodes, which may be sixty electrodes arranged in columns of fifteen, i.e., as a first column of electrodes 401a . . . 401o, a second column of electrodes 403a . . . 403o, a third column of electrodes 405a . . . 405o, and a fourth column of electrodes 407a . . . 407o. Although sixty electrodes are illustrated and described, any number of electrodes may be utilized. Each electrode of the first electrode array may be 3 mm long and 1 mm wide. The second electrode array 500 may include eight electrodes, that is, electrode 501a, 501b, 501c, 501d, 501e, 501f, 501g, and 501h. Each electrode of the second electrode array 500 may be 3 mm long and 3 mm wide. Although described and illustrated as including an array of electrodes, the EIT system 450 may include any number of electrodes or group of electrodes, for example, one or more electrodes. For example, in aspects, one or more of the first electrode array 400 and/or the second electrode array 500 includes a number of electrodes ranging between eight electrodes and one hundred and twenty eight electrodes.

In aspects, the electrodes of the first electrode array 400 may be grouped into four distinct sets of ten electrodes (e.g., first set 410, second set 420, third set 430, and fourth set 440), with each set of ten electrodes being paired with eight electrodes of the second electrode array 500. Depending on the number of inputs included in the EIT system 450, the electrodes of the first electrode array 400 and/or the electrodes of the second electrode array 500 may be multiplexed to match the number of inputs desired.

The EIT system 450 is configured to apply an electrical current across the first electrode array 400 and the second electrode array 500, or only the first electrode array 400, for example, at a frequency from about 10 kHz to about 80 kHz, although other frequencies are contemplated, for example in the range of 100 Hz to 1 MHz. The voltage difference across the first electrode array 400 and the second electrode array 500, or the first electrode array 400 only, is measured and the processor 451 calculates an electrical impedance of the tissue based on the measured voltage difference. The EIT system 450 is configured to apply an electrical current across any two electrodes of one or both of the first electrode array 400 and/or the second electrode array 500 and calculate the voltage difference between any two electrodes of one or both of the first electrode array 400 and/or the second electrode array 500. Thus, the EIT system 450 may be configured to apply current and measure voltage across electrodes of a single electrode array (e.g., first electrode array 400) or apply current and measure voltage across electrodes of a two electrode arrays (e.g., electrodes of first electrode array 400 and electrodes of second electrode array 500 located on an opposing jaw).

The processor 451 then generates an electrical impedance tomography reconstruction 600 (FIG. 6) based on the calculated electrical impedance for display on display 12 (FIG. 1) of computing device 100 or for other processing by computing device 100 (e.g., alarms and notifications). The generated electrical impedance tomography reconstruction can be visualized in 3D (FIG. 6) or as 2D images where regions of high, low, or specific conductivity values can be highlighted to show cancerous regions, demarcate different tissues, demarcate health states, and/or detect critical structures. The EIT data set or reconstruction may include reactive inclusions as well (e.g., those with permittivity). In aspects, the processor may detect noise within the impedance data and filter noisy patterns prior to generating the electrical impedance tomography reconstruction 600.

With reference to FIG. 6, an electrical impedance tomography reconstruction 600 is shown which includes conductive inclusions 605 and resistive inclusions 607 reconstructed based on the electrical impedance data. The conductive inclusions 605 may be optically distinguished from the resistive inclusions 607 by being displayed within the electrical impedance tomography reconstruction 600 in a different color or shading, though other non-optical or non-visual feedback may be additionally or alternatively incorporated. The data provided to form the electrical impedance tomography reconstruction 600 may also be used by processor to detect tumor margins for display within the electrical impedance tomography reconstruction 600.

The following Examples illustrate embodiments of the present disclosure. These Examples are intended to be illustrative only and are not intended to limit the scope of the present disclosure.

### Experimentation and Simulation

Simulated experiments were used to determine an ideal sensitivity threshold, evaluate modeling/noise factors (impedance noise, error in assumed stapler separation), and assess the best way to analyze the 3D EIT reconstructions. Further, both a single/bottom-only electrode array configuration (e.g., first electrode array 400 only) and a dual/top-and-bottom electrode array configuration (e.g., both the first electrode array 400 and the second electrode array 500) were evaluated in all experiments. This 1-sided versus 2-sided electrode array comparison is useful in developing a clinically translatable stapler/EIT combination device, as the two configurations will have different depth sensitivities. The technique was evaluated on ex vivo bovine samples for visualization and delineation of adipose and muscle tissues. Accurate tissue reconstructions are a good indication of the robustness of the approach in practice.

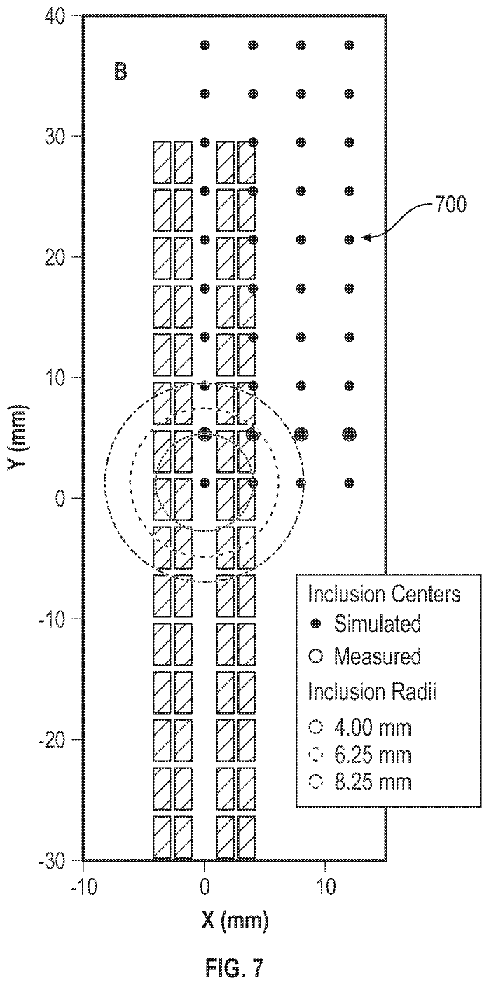

Two circuit boards containing arrays of gold-plated electrodes were used to emulate the two sides of the stapler or grasper. One side had sixty 3×1 mm closely-spaced electrodes (bottom side), while the opposite (top) side had eight 3×3 mm electrodes spaced further apart. Simulated grid marks 700 (FIG. 7) using 4 mm spacing were used to aid in segmenting the phantoms. Although sixty electrodes are illustrated and described, any number of electrodes may be utilized. Both the first and second electrode arrays 400, 500 span a 59×8 mm area, an appropriate size to fit on the flat inner plates of a surgical stapler.

During experimentation, the EIT data was collected using a 20-channel custom EIT system. Because of the limited channel count, data was collected from a subset of electrodes by multiplexing the 20 channels to 4 sets of 18 electrodes (e.g., first set 410, second set 420, third set 430, and fourth set 440 shown in FIG. 4). Each set uses all eight electrodes from the top side and 10-electrodes from a single column on the bottom side. Therefore, only 48 of the 60 bottom electrodes are used. The EIT system collects exhaustive IIVV patterns of impedance data on the 18 electrode sets. Each measurement is referred to as an IIVV pattern where II represents the current injection electrode pair (11,12) and VV represents the differential voltage sensing pair of electrodes (V1,V2). All acquired data was pre-filtered to remove noisy patterns prior to image reconstruction. Data was recorded at 4 frequencies, 10, 20, 40, and 80 kHz.

Forward Problem: The forward problem computes the voltages on the electrodes given a specified injected current on electrodes and internal conductivity distribution. A Complete Electrode Model (CEM) was used, which realistically accounts for the contact impedance and shunting of electrodes. The approach utilized a 3D Finite Element Method (FEM) implementation using linear basis elements on a tetrahedral mesh construct for this scenario. For example, the mesh may be constructed via DistMesh mesh generator to produce 2D triangulations of the outer boundary with encoded electrodes and Gmsh mesh generator to produce the full 3D mesh. The stapler separation was in the z-direction with the short and long stapler axes given by the x-axis and y-axis, respectively (see FIG. 7). The circuit boards were assumed to have a constant separation distance of 10 mm (at any x/y-position). The open-domain (open in the x- and y-direction) was modeled by extending the mesh until the impedances were unaffected by the boundary. Overall, the mesh was extended 50 mm in the x-direction and 30 mm in the y-direction. The mesh density near the electrodes and in the background region were empirically tuned to produce accurate impedance values. The overall mesh had ˜160k nodes and 830k elements. Ill-shaped elements were eliminated via Gmsh's element-shape optimization routine.

Inverse Problem: A standard Gauss-Newton algorithm is employed using Laplace-smoothing Tikhonov regularization producing 3D conductivity images. The aim is to minimize the following objective function:

$${arg min}_{δσ} {‖ J δσ - ( {v}_{Tst} - {v}_{Ref} ) ‖}^{2} + λ {‖ L ( {σ}_{0} + δσ ) ‖}^{2} \qquad ( 1 )$$

where δσ is the conductivity update, σ₀ is the initial estimate, J is the Jacobian, v_{Tst} and v_{Ref} are the test and reference voltages, L is the regularization matrix, and λ is the Tikhonov regularization factor. The Jacobian is solved using lead field theory, which in a physics-based manner gives appropriate sensitivity to each electrode/impedance measurement due to its size and geometry. The least squares solution to (1) is:

$$δ \tilde{σ} = {( {J}^{T} J + λ {L}^{T} L )}^{- 1} ( {J}^{T} Δ v - λ {L}^{T} L {σ}_{0} ) \qquad ( 2 )$$

where J and Δν are the Jacobian and voltage difference data. The process can be iterated to obtain absolute reconstructions.

A dual-mesh method is used which maps the fine FEM mesh to a coarse inverse mesh. This step addresses numerical concerns and also allows for a convenient framework to separate inverse voxels with sufficient sensitivity for imaging from those that are not sensitive enough. The dual mesh in this study is based on a rectilinear grid of voxels 36×40×8, resulting in voxels of size 3.0×3.0×1.3 mm. The Jacobian on the coarse mesh, J^{C}, is therefore of size number of IIVV patterns (Nᵢᵢᵥᵥ) by the number of inverse voxels (NI). The method relies on choosing a threshold, S, and calculating the vector of relative sensitivities across all IIVV patterns:

$${j}_{S , j} = {max}_{1 \leq i \leq Niivv} \vert {J}_{ij}^{C} \vert / {max}_{1 \leq i , j \leq Niivv , {N}_{I}} \vert {J}_{ij}^{C} \vert \qquad ( 3 )$$

Inverse voxels less than the threshold are then grouped into a so-called mega-node, yielding a single reconstructed conductivity value for all corresponding voxels. This approach is particular useful in applications such as this where the domain is open, electrodes are only on a portion of the surface, and there are large regions with very limited sensitivity.

Simulated and measured phantom experiments were performed that evaluated the ability to detect single inclusions of varying size, contrast and location. The inclusion locations were based on a grid viewable on the electrode array. A complete list of the inclusion parameters considered is given in Table I, which results in a total of 720 simulated samples. There were 25 measured phantom tests, a subset from Table I, and an explicit list is provided in Table II.

TABLE I
Simulated Inclusion parameters.

| Data Type | Values | |
|---|---|---|
| X-offset (from center) | 0, 4, 8, 12 | mm |
| Y-offset (from center) | 0 to 36 in 4 mm steps | |
| Contrast | 10, 20, 30, 40, 50, 100% | |
| Size | 8, 12.5, 16.5 | mm |

| | |
|---|---|
| Voltage Noise | 0.1, 0.2, 0.5, 1, 2, 5% |
| (standard deviation) | |

TABLE II
Measured Inclusion parameters.

| | | X-offset | | |
|---|---|---|---|---|
| Inclusion | Contrast | (mm) | | N |
| Small (8 mm) | 10*, 20*, 30, | 0 | mm | 10 |
| | 40*, 50, 100% | | | |
| Small (8 mm) | 50%, 100% | 0, 4, 8 | mm | 6 |
| Medium (12.5 mm) | 50, 100% | 0, 4, 8, 12 | mm | 8 |
| Large (16.5 mm) | 100% | 0 | mm | 1 |

*implies there were repeat measurements of these samples.

The measured inclusion tests were performed by first constructing 10 mm thick agar gel molds that extended past the electrodes in each direction. The agar gel recipe was 3% Agar Powder (Hoosier Hill Farm, Ft. Wayne, IN), 1% Sorbitol in a 0.1 S/m saline solution. After mixing and heating, the liquid gel was poured into a test cell to obtain the gel conductivity and into a mold 10 mm thick. The resulting gel was measured to be 0.115 S/m. The inclusions were cut out of the gel using metal tubes of 8, 12.5, and 16.5 mm diameters and filled with saline corresponding to the desired contrast. Drops of saline and a 700 g brass weight were used to ensure good electrode contact between the electrode arrays and gel.

Two parameters needed to be selected to optimize reconstructions; the Tikhonov/regularization parameter (λ) and sensitivity threshold(S). The Tikhonov parameter was chosen to maximize the resolution while limiting artifacts and was heuristically selected through qualitative evaluation over a set of difference reconstructions. The sensitivity threshold was chosen to maximize the area-under-the-curve (AUC) from receiver operator characteristic (ROC) curve analysis over all the samples in the simulated database. The AUC analysis is performed on a x/y-slice basis, i.e. all voxels from all reconstructions from a given slice are used. On these voxels the EIT values are compared to labels (background or inclusion).

The simulated database of parameters additionally allowed the study of the effects of location, size, contrast, and the effect of varying voltage noise and the reference height. Varying the reference height is a modeling-error test that evaluates the effect of incorrectly modeling the distance between the top and bottom electrodes. In all voltage noise analyses, 20 repetitions were sampled using unbiased, normally distributed noise of the specified percent of the ideal voltage values (0.1-5%).

Throughout the analysis of the simulated and measured inclusions reconstructions using top and bottom electrodes or bottom-only electrodes were considered. The top-side electrodes were included to improve depth of sensitivity, but do add complexity to the system and may increase the sensitivity to the top-bottom array spacing.

Simulated Inclusion Tests: The simulated inclusion tests were used to 1) determine a best sensitivity threshold and x/y-slice for analysis and 2) evaluate the sensitivity/effects of voltage noise, reference height mismatch, inclusion contrast, size, and x-offset. Reference height refers to the mesh height (or electrode array separation distance) used to calculate the reference voltages, v_{Ref}.

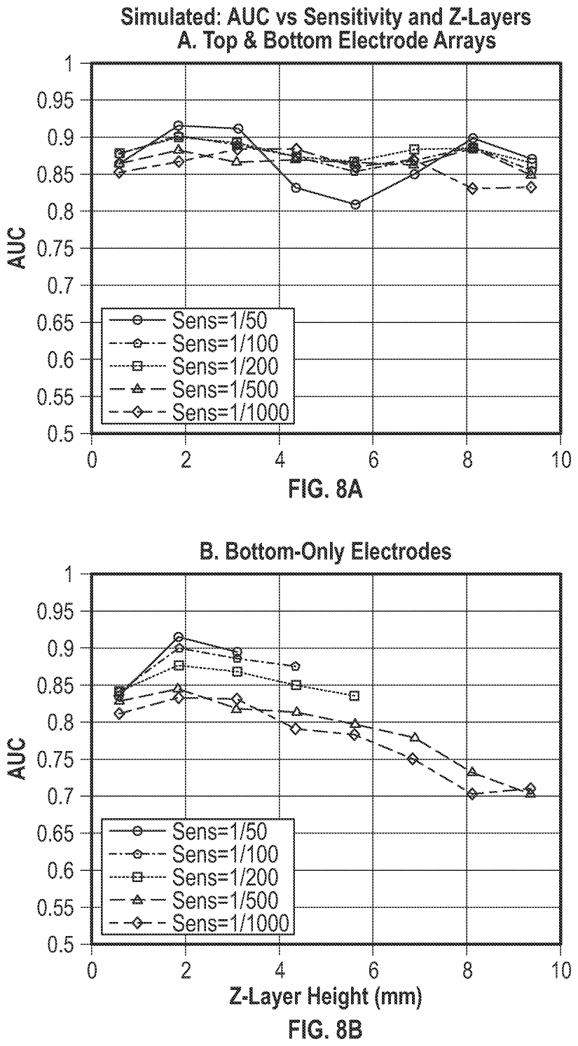

AUC analysis of top and bottom electrodes (FIG. 8A) and bottom-only (FIG. 8B) versus x/y-slice (z-layer) and sensitivity threshold revealed that 1) the best z-slice occurs at the 2nd layer from the bottom and 2) the 1/50th sensitivity threshold works best regardless of which electrodes are used. There are two interesting aspects of the curves in FIGS. 8A and 8B that are related to the shape of the region of sensitivity. First, in FIG. 8A the region of sensitivity is large near the top and bottom electrodes but narrows in the center. This narrowing becomes an increasing factor as the sensitivity threshold increases, i.e. notice the two humps with central minima in FIG. 8A for higher sensitivity thresholds 1/50 and 1/100. Similarly, when only using the bottom electrodes the region of sensitivity significantly reduces with higher thresholds, i.e. notice the fewer z-layers for increasing thresholds in FIG. 8B. All subsequent reconstructions use a 1/50 threshold and are analyzed at the 2nd z-slice.

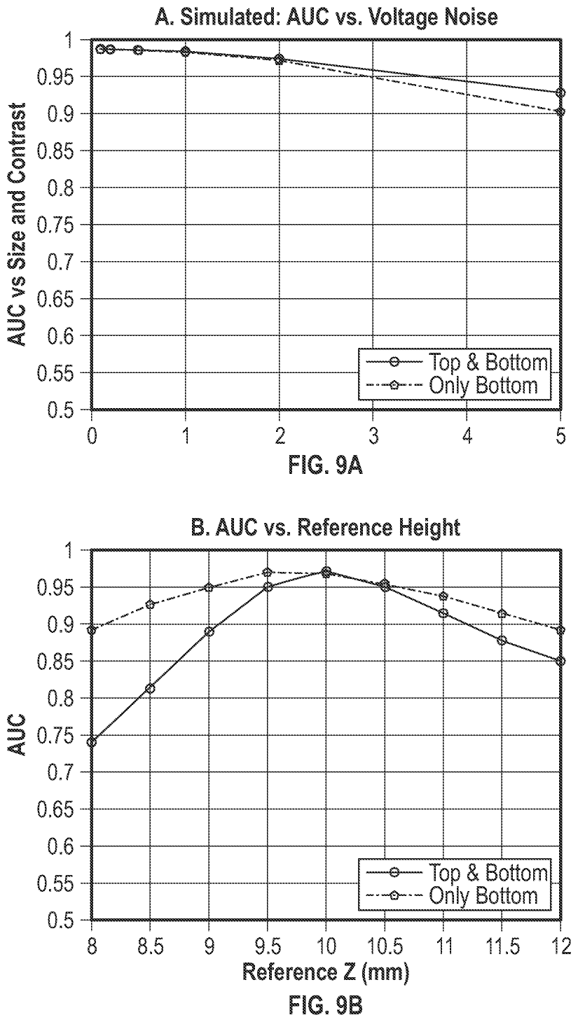

The effect on AUC from voltage noise (FIG. 9A) and reference height (FIG. 9B) reveal minimal loss in accuracy for noise up to 1% and that bottom-only reconstructions are much less sensitivity to height mismatches. Specifically, the AUC stays ˜0.98 up to 1% voltage error and only drops to 0.97 by 2% regardless of which electrodes are used. There is only a noticeable difference between electrodes used at 5% noise (0.93 bottom and top and 0.90 for bottom only). In terms of the reference height, if there is a 1 mm error in the assumed separation (9 mm or 11 mm) the top and bottom electrode results drop to 0.90-0.91 compared to 0.94-0.95 for bottom-only electrodes, where the maximum AUC is 0.97 regardless of electrodes used and occurs when there is no reference-height error.

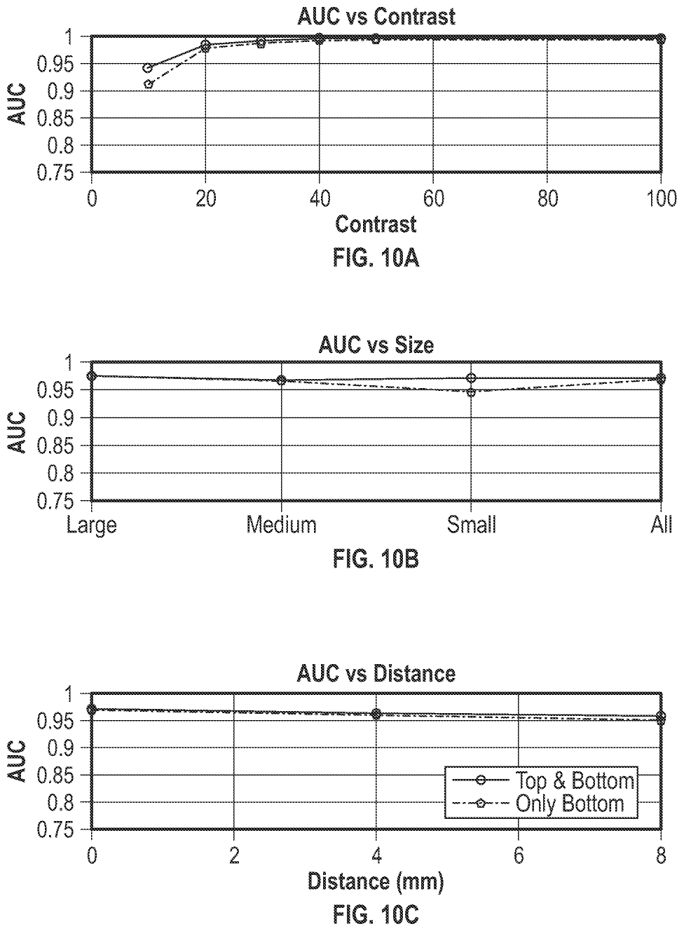

The last factors considered are the AUC versus the inclusion contrast, size, and distance (FIGS. 10A-10C). Here, distance is the x/y-distance from the electrodes to the center of the inclusion. The contrast appeared to be the factor with the largest effect. Bottom-only and top and bottom electrode AUCs reduced to 0.91 and 0.94, respectively at a 10% contrast compared to peak values of 0.99 achieved at 30% and greater contrast. The size and distance variations resulted in smaller effects, but in each case the bottom-only results were slightly worse compared to top and bottom electrodes. For size, bottom-only electrodes yielded an AUC of 0.94 compared to 0.97, and for distance there was a 1% drop for distances of 4 and 8 mm.

Based on the simulation tests, the optimal region for analysis lies within the 2^{nd} x/y-slice (z-slice) (1.25 mm from the bottom electrodes) and when using a sensitivity threshold of 1/50 extends essentially to the edge of the electrodes. Although it is necessary to model the current flowing beyond/outside the arrays, it is intuitive that the most accurately imaged region falls directly between the arrays. The optimal z-slice is somewhat less intuitive. Electrode artifacts are often found in EIT, so the layer closest to the 60-electrode side may be more challenging to image. Likewise, 1) the quality may decrease in the middle of the domain where the sensitivity is least and 2) the x/y-resolution may be somewhat limited towards the top side as there are only 8 electrodes. Lastly, the bovine tests appear to imply that the region for analysis should be further reduced in the y-direction to roughly half the total extent. This was unexpected based on simulated tests and measured gel tests.

While the disclosure is presented with respect to surgical instruments including handle assemblies, it should be understood the principles of the disclosure may also be applied to robotic surgical systems that do not technically include handle assemblies. The disclosed GUI scheme may be adapted in teleoperation, robotic surgery systems. It will be understood that various modifications may be made to the embodiments of the presently disclosed staplers. Therefore, the above description should not be construed as limiting, but merely as exemplifications of embodiments. Those skilled in the art will envision other modifications within the scope and spirit of the present disclosure.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A surgical system comprising:
a surgical stapler including an end effector, the end effector including a first jaw having an anvil and a second jaw having a stapler cartridge; and
an electrical impedance tomography system including:
an electrode array including a plurality of electrodes operably coupled to the first jaw; and
a processor configured to:
control an application of electrical current across electrodes of the electrode array;
measure a voltage difference across electrodes of the electrode array;
calculate electrical impedance based on the measured voltage difference; and
generate an electrical impedance tomography reconstruction based on the calculated electrical impedance.

2. The surgical system of claim 1, wherein the electrode array includes a plurality of electrodes.

3. The surgical system of claim 1, wherein each electrode of the electrode array is about 3 mm long and about 1 mm wide.

4. The surgical system of claim 1, wherein the electrodes of the electrode array are arranged in four columns and fifteen rows.

5. The surgical system of claim 1, wherein the electrodes of the electrode array are grouped into four distinct sets of ten electrodes.

6. The surgical system of claim 1, wherein the processor is configured to filter the calculated electrical impedance by removing noisy patterns prior to generating the electrical impedance tomography reconstruction.

7. The surgical system of claim 1, wherein the generated electrical impedance tomography reconstruction includes conductive inclusions and resistive inclusions, wherein the conductive inclusions are optically distinguishable from the resistive inclusions.

8. The surgical system of claim 1, wherein the processor is configured to detect tumor margins based on the electrical impedance tomography reconstruction.

9. The surgical system of claim 1, wherein the processor is configured to control an application of the electrical current at a frequency from about 10 kHz to about 80 KHz.

10. A powered surgical instrument comprising:
an end effector including a first jaw and a second jaw;
an electrical impedance tomography system including:
a first electrode array including a plurality of electrodes operably coupled to the first jaw;
a second electrode array including a plurality of electrodes operably coupled to the second jaw; and
a processor configured to:
control an application of an electrical current across the first electrode array and the second electrode array;
measure a voltage difference across the first electrode array and the second electrode array;
calculate electrical impedance based on the measured voltage difference; and
generate an electrical impedance tomography reconstruction based on the calculated electrical impedance.

11. The powered surgical instrument of claim 10, wherein the first electrode array includes sixty electrodes and the second electrode array includes eight electrodes.

12. The powered surgical instrument of claim 10, wherein each electrode of the first electrode array is about 3 mm long and about 1 mm wide and each electrode of the second electrode array is about 3 mm long and about 3 mm wide.

13. The powered surgical instrument of claim 10, wherein the electrodes of the first electrode array are arranged in four columns and fifteen rows and the electrodes of the second electrode array are arranged in two columns and four rows.

14. The powered surgical instrument of claim 10, wherein at least a portion of the plurality of electrodes of the first electrode array are grouped into four distinct sets of ten electrodes, each set of ten electrodes being paired with eight electrodes of the second electrode array.

15. The powered surgical instrument of claim 10, wherein the processor is configured to filter the calculated electrical impedance by removing noisy patterns prior to generating the electrical impedance tomography reconstruction.

16. The powered surgical instrument of claim 10, wherein the generated electrical impedance tomography reconstruction includes conductive inclusions and resistive inclusions, wherein the conductive inclusions are optically distinguishable from the resistive inclusions.

17. The powered surgical instrument of claim 10, wherein the processor is configured to detect tumor margins based on the electrical impedance tomography reconstruction.

18. The powered surgical instrument of claim 10, wherein the processor is configured to control an application of the electrical current across the first electrode array and the second electrode array at a frequency from about 10 kHz to about 80 kHz.

19. A surgical system comprising:
a powered surgical instrument including a first jaw and a second jaw;
an electrical impedance tomography system including:
a first electrode array including a plurality of electrodes operably coupled to the first jaw;
a second electrode array including a plurality of electrodes operably coupled to the second jaw; and
a processor configured to:
control an application of an electrical current across the first electrode array and the second electrode array;
measure a voltage difference across the first electrode array and the second electrode array;
calculate electrical impedance based on the measured voltage difference; and
generate an electrical impedance tomography reconstruction based on the calculated electrical impedance; and
a display operably couped to the electrical impedance tomography system and configured to display the electrical impedance tomography reconstruction.

20. The surgical system of claim 19, wherein the processor is configured to control an application of the electrical current across the first electrode array and the second electrode array at a frequency from about 10 kHz to about 80 kHz.
